(19) United States
(12) Patent Application Publication
(10) Pub. No.: US 2021/0379273 A1
(43) Pub. Date: Dec. 9, 2021

(54) BRIDGE DRESSING WITH FLUID MANAGEMENT

(71) Applicant: KCI Licensing, Inc., San Antonio, TX (US)

(72) Inventors: Christopher Brian LOCKE, Bournemouth (GB); Richard Daniel John COULTHARD, Verwood (GB); Justin Alexander LONG, Lago Vista, TX (US); Thomas Alan EDWARDS, Hampshire (GB); Timothy Mark ROBINSON, Shillingstone (GB)

(21) Appl. No.: 17/046,023

(22) PCT Filed: Mar. 1, 2019
(86) PCT No.: PCT/US2019/020308

§ 371 Date: Oct. 8, 2020

Related U.S. Application Data
(60) Provisional application No. 62/655,576, filed on Apr. 10, 2018.

(51) Int. Cl. A61M 1/00; A61F 13/00

(52) U.S. Cl. CPC A61M 1/913; A61M 1/915; A61F 13/00068

(57) ABSTRACT

An evaporative bridge dressing that may be used with negative-pressure treatment of tissue. The evaporative bridge dressing may have one or more fluid transfer layers comprised of high-density wicking material enclosed between layers of film having high moisture-vapor transfer rates to manage liquid storage and pressure drop. The evaporative bridge may have an absorbent in some embodiments. An evaporation channel may be disposed adjacent to or combined with the evaporative bridge. A means for measuring pressure across the evaporative bridge may include a feedback path. A support means may reduce or prevent collapse of one or more of the evaporative bridge, the evaporation channel, the feedback path.

### RELATED APPLICATION

This application claims the benefit, under 35 U.S.C § 119(e), of the filing of U.S. Provisional Patent Application Ser. No. 62/655,576, entitled “BRIDGE DRESSING WITH FLUID MANAGEMENT,” filed Apr. 10, 2018, which is incorporated herein by reference for all purposes.

### TECHNICAL FIELD

The invention set forth in the appended claims relates generally to tissue treatment systems and more particularly, but without limitation, to bridge dressings with fluid management for use with negative-pressure treatment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

In some embodiments, an apparatus for treating tissue with negative pressure may comprise a dressing, an evaporative fluid bridge, a fluid interface, and a fluid conductor. The apparatus may be beneficial for various modes of treatment and for various types of tissue sites and may be particularly advantageous for use with compression bandages on shallow, highly exuding wounds, such as venous leg ulcers.

The dressing may comprise a contact layer comprised of a perforated silicone, with or without adhesive, a non-adherent polyethylene film, or ethylene-vinyl acetate mesh. The contact layer can provide adhesion and allow the dressing to be repositioned without loss of adhesion. The dressing may additionally comprise an occlusive, adhesive-coated polyurethane cover layer. One or more high-density wicking layers can be enclosed between the contact layer and the cover layer. The dressing may have no absorbent in some embodiments. The wicking layers can draw exudate and other fluid from a tissue site into the dressing and transfer the fluid to the fluid bridge. The dressing may be configured for wrapping around a limb in some embodiments. For example, some embodiments of the dressing may have flaps or wings configured to be wrapped around a leg or arm.

The fluid bridge may be fluidly coupled to the dressing. In some embodiments, the fluid bridge may comprise a lower layer of adhesive-coated polyurethane film, and a top layer of highly breathable (high MVTR) polyurethane film without an adhesive coating. One or more intermediate layers of low-profile, high-density wicking and manifolding agents may be disposed between the lower layer and the top layer. The intermediate layers can draw fluid through the bridge toward the fluid interface. An additional intermediate layer of hydrophobic wicking material or partial layer patterned or die cut may be used to facilitate manifolding of negative pressure and transporting liquid.

The fluid interface can provide a low-profile, comfortable aperture to fluidly connect the fluid bridge to the fluid conductor, which can be fluidly coupled to a source of negative pressure. In some embodiments, the fluid interface may be manufactured from a flexible polymer, such as polyvinyl chloride (free of diethylhexyl phthalate). In some embodiments, the fluid conductor may be a tube also manufactured from a flexible polymer, such as polyvinyl chloride (free of diethylhexyl phthalate). The fluid conductor may have a single lumen in some embodiments. For example, the fluid conductor may have a single lumen with a consistent inner dimension of about 2.4 millimeters. In other embodiments, the fluid conductor may have multiple lumens, which may be suitable for use with feedback mechanisms.

An evaporation channel may be positioned proximate to the top layer of high-MVTR film in some embodiments. For example, the evaporation channel may comprise or consist essentially of an additional film layer that can contain an air stream along a length of the fluid bridge.

An air-flow control system can generate a therapeutic negative pressure to treat tissue and a flow of air to assist with the evaporation of collected fluids. In some embodiments, the flow control system may use a single pump with a valve switching flow for both flow streams in some embodiments. For example, exhaust from the pump may be channeled into an evaporation channel, and a valve can be used to determine if the input to the pump is from the fluid bridge or from local atmosphere. The flow control system may be mounted on an end of the fluid bridge furthest away from the dressing, and may be pneumatically connected so that a pump or other source of negative pressure can draw air and exudate through the length of the fluid bridge. The flow control system may contain batteries in some examples to allow a patient to be mobile. Some batteries may be recharged by an inductive-coupled system to avoid having a socket on the exterior.

Operator feedback can be provided with LEDs, haptic vibration alerts, or other means to discretely draw an operator's attention. Additionally or alternatively, wireless communications can be used to communicate more detailed information to a base station or a smart device. For example, BLUETOOTH LOW ENERGY (BLE) is generally optimized for short-range, low-power communications and may be particularly suitable for some examples.

Some embodiments may comprise a means to measure pressure at a distal end of the fluid bridge, which may be in close proximity to the dressing. For example, some embodiments may comprise a feedback channel that is fluidly isolated from and substantially parallel to the fluid bridge. A controller may be configured to compare this pressure reading with one taken at the source of negative pressure and determine one or more operating conditions, such as pressure drop across the fluid bridge or a level of saturation in the fluid bridge. A controller may also be configured to take action based on the operating conditions. For example, a feedback path may be pneumatically coupled to the distal end of the fluid bridge, and a sensor may be pneumatically coupled to the feedback path. In some embodiments, a filler medium, a textured surface, or other support means may ensure that the feedback path remains open under external pressure.

In use, the dressing may be applied directly to a tissue site, and the bridge may be adhered comfortably to the dressing. Alternatively, the bridge may be adhered to the dressing before the dressing is applied to a tissue site. The fluid interface and the fluid conductor may be supplied attached or unattached to the bridge. In some treatment modes, at least some portion of the dressing, the bridge, or both may be covered by a compression means, such as a bandage or compression garment. The compression means may be hydrophobic or hydrophilic and should be breathable so as to not prevent the exchange of air flow with the evaporative bridge. The compression means may be supplied as a component of the apparatus, or may be sourced separately.

More generally, some embodiments of an apparatus for treating a tissue site in a negative-pressure environment may comprise an envelope and a fluid transfer bridge enclosed by the envelope. The envelope may comprise a vapor-transfer surface, a first transfer channel, and a second transfer channel. An evaporation channel may be disposed adjacent to the vapor-transfer surface. A range of about 250-5000 grams per square meter per twenty-four hours may be a suitable rate of moisture transfer for some embodiments. In some embodiments, the fluid transfer bridge may comprise an absorbent, such as a super-absorbent polymer. The absorbent may be disposed between two wicking layers in some examples.

Additionally or alternatively, at least one of the envelope and the evaporation channel may comprise a support means, such as a filler medium, a textured interior surface, or embossed surface.

In some embodiments, an apparatus for treating a tissue site with negative pressure may comprise an envelope defining a fluid chamber, and an absorbent within the fluid chamber. The envelope may comprise at least one vapor-transfer surface, a first transfer channel, and a second transfer channel. An evaporation channel may be disposed adjacent to the vapor-transfer surface. A negative-pressure port of a pump may be fluidly coupled to the second transfer surface, and a positive-pressure port may be fluidly coupled to the evaporation channel in some examples. A controller may be configured to operate a valve to selectively couple the negative-pressure port and the positive-pressure port to at least one of the fluid chambers, the evaporation channel, and ambient air.

In some embodiments, an apparatus for treating a tissue site with negative pressure may comprise an envelope having at least one vapor-transfer surface and defining an elongated fluid chamber having a proximal end and a distal end. A fluid transfer bridge can be disposed in the fluid chamber. The apparatus may additionally comprise a means for measuring pressure adjacent to the distal end of the fluid chamber.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a schematic view of an example of a bridge dressing that may be associated with some embodiments of the therapy system of FIG. 1;

FIG. 3 is a schematic section of the bridge dressing of FIG. 2, illustrating additional details that may be associated with some embodiments;

FIG. 4 is an assembly view of an example of the bridge dressing of FIG. 2;

FIG. 5 is a plan view of a contact layer that may be associated with some embodiments of the bridge dressing of FIG. 4;

FIG. 6 is a chart illustrating pressure drop performance that may be associated with some features of the bridge dressing of FIG. 2;

FIG. 7 is a schematic section of another example of the bridge dressing of FIG. 2, illustrating additional details that may be associated with some embodiments;

FIG. 8 is an assembly view of an example of a fluid bridge that may be associated with some embodiments of the therapy system of FIG. 1;

FIG. 9 is a schematic section of another example of the fluid bridge of FIG. 2, illustrating additional details that may be associated with some embodiments.;

FIG. 10A and FIG. 10B are schematic section diagrams illustrating other features that may be associated with some embodiments of the fluid bridge of FIG. 1;

FIG. 11 is a schematic diagram of an example configuration of an evaporation channel that may be associated with the bridge dressing of FIG. 2;

FIG. 12 is a schematic section of the evaporation channel of FIG. 11;

FIG. 13 illustrates another example configuration of the bridge dressing of FIG. 2;

FIG. 14 is a functional block diagram illustrating additional details that may be associated with some embodiments of the therapy system of FIG. 1;

FIG. 15 is a schematic diagram illustrating additional details that may be associated with the operation of therapy system of FIG. 14;

FIG. 16 is a schematic diagram illustrating additional details that may be associated with the operation if the therapy system of FIG. 14;

FIG. 17 is a functional block diagram illustrating additional details that may be associated with some embodiments of the therapy system 100;

FIG. 18 is a schematic diagram illustrating additional details that may be associated with the operation of the therapy system of FIG. 17;

FIG. 19 is a schematic diagram illustrating additional details that may be associated with the operation of the therapy system of FIG. 18;

FIG. 20 is a schematic view of another example of the therapy system of FIG. 1; and

FIG. 21 is a schematic section of a fluid bridge that may be associated with some embodiments of the therapy system of FIG. 20.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

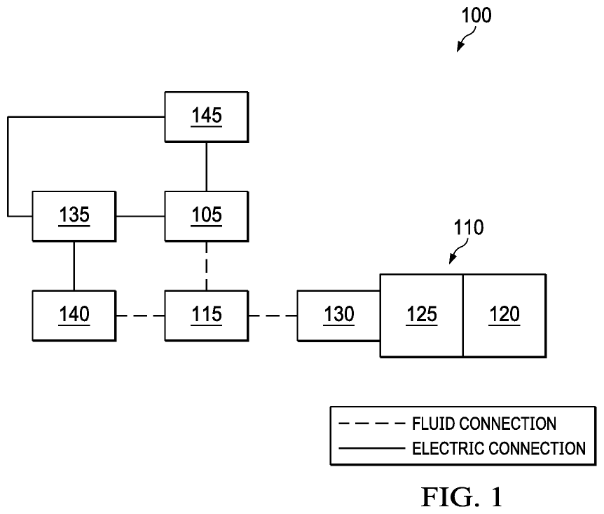

FIG. 1 is a simplified functional block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments. A fluid bridge 130 may fluidly couple the dressing 110 to other components, such as the negative-pressure source 105.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 135. Additionally, the therapy system 100 may include sensors to measure operating parameters and provide feedback signals to the controller 135 indicative of the operating parameters. As illustrated in FIG. 1, for example, the therapy system 100 may include a first sensor 140 and a second sensor 145 coupled to the controller 135.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 135 and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 135 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. In some embodiments, the negative-pressure source 105 may be powered by batteries to facilitate mobility, and the batteries may be recharged by an inductive charging system in some examples. A miniature air pump may be suitable for some applications. For example, Koge Micro Tech Co., Ltd. manufactures a disc pump that can provide suitable pressure while providing a low profile and low noise.

“Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mmHg (−667 Pa) and −500 mmHg (−66.7 kPa). Common therapeutic ranges are between −50 mmHg (−6.7 kPa) and −300 mmHg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 135, may be a microprocessor or computer programmed to operate one or more components of the therapy system 100, such as the negative-pressure source 105. In some embodiments, for example, the controller 135 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 135 is also preferably configured to receive one or more input signals, such as a feedback signal, and programmed to modify one or more operating parameters based on the input signals. In some embodiments, the controller 135 can provide alerts or other indicators as feedback to an operator. For example, the controller 135 may be configured to activate light-emitting diodes or haptic vibration to discretely alert an operator to certain conditions. Additionally or alternatively, audio alerts may be activated.

Sensors, such as the first sensor 140 and the second sensor 145, are generally known in the art as any apparatus operable to detect or measure a physical phenomenon or property, and generally provide a signal indicative of the phenomenon or property that is detected or measured. For example, the first sensor 140 and the second sensor 145 may be configured to measure one or more operating parameters of the therapy system 100. In some embodiments, the first sensor 140 may be a transducer configured to measure pressure in a pneumatic pathway and convert the measurement to a signal indicative of the pressure measured. In some embodiments, for example, the first sensor 140 may be a piezo-resistive strain gauge. The second sensor 145 may optionally measure operating parameters of the negative-pressure source 105, such as a voltage or current, in some embodiments. Preferably, the signals from the first sensor 140 and the second sensor 145 are suitable as an input signal to the controller 135, but some signal conditioning may be appropriate in some embodiments. For example, the signal may need to be filtered or amplified before it can be processed by the controller 135. Typically, the signal is an electrical signal, but may be represented in other forms, such as an optical signal. In some examples, signals may be transmitted wirelessly to the controller 135. Additionally or alternatively, signals may be transmitted to a base station or smart device. For example, Bluetooth LE may be a suitable protocol as it is optimized for short-range, low-power wireless communications.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise a means to transfer fluid. For example, the tissue interface 120 may comprise or consist essentially of a fluid transfer member, such as a manifold member, a wicking member, or some combination of manifold and wicking members. A manifold member in this context generally includes material, substances, or structures that provide pathways adapted to collect or distribute fluid across a tissue site under pressure. A wicking member generally includes material, substances, or structures that can move liquid by capillary action. In some illustrative embodiments, the pathways of a manifold or wicking member may be interconnected to improve distribution or collection of fluids across a tissue site.

In some illustrative embodiments, a manifold may be a porous material having interconnected cells. For example, open-cell foam generally includes pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

Some textiles may also be suitable as a fluid transfer member. For example, woven and non-woven textiles are generally porous, making them suitable as a manifold in some embodiments. Some textiles may additionally or alternatively be configured to transfer fluid through wicking action. In general, a textile includes any cohesive network of natural or synthetic fibers. For example, fibers may be woven, knitted, knotted, pressed together, or otherwise bonded to form a textile. Sheets or webs of fibers that are bonded together by entangling fibers mechanically, thermally, or chemically are generally classified as a non-woven textile. More broadly, though, a non-woven textile may include any sheet or layer of fibers which are neither woven nor knitted, such as felt, for example.

In some embodiments, a fluid transfer member may be a composite textile having a hydrophobicity that varies from a first side to a second side. For example, the hydrophobicity may increase from an acquisition surface to a distribution surface. In some examples, a fluid transfer member may be a non-woven textile having an acquisition surface that is hydrophilic and a distribution surface that is hydrophobic. In some embodiments, a fluid distribution surface may include hydrophobic fibers oriented substantially within a plane of the surface. A fluid acquisition surface may include hydrophilic fibers oriented substantially normal to a plane of the surface. More specifically, in some example embodiments, a fluid transfer member may comprise or consist essentially of a dual-layer non-woven textile, such as a through-air bonded web of dry polyester and hydrophilic, profiled polyester and bi-component fibers. Suitable products may include the DRYWEB TDL2 acquisition and distribution layer from LIBELTEX, or the SLIMCORE TL4 acquisition and distribution layer from LIBELTEX, for example.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 135 may receive and process data from one or more sensors, such as the first sensor 140. The controller 135 may also control the operation of one or more components of the therapy system 100 to manage the pressure delivered to the tissue interface 120. In some embodiments, controller 135 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 135. The target pressure may vary from tissue site to tissue site based on the type of tissue forming a tissue site, the type of injury or wound (if any), the medical condition of the patient, and the preference of the attending physician. After selecting a desired target pressure, the controller 135 can operate the negative-pressure source 105 in one or more control modes based on the target pressure and may receive feedback from one or more sensors to maintain the target pressure at the tissue interface 120.

In some embodiments, the controller 135 may have a continuous pressure mode, in which the negative-pressure source 105 is operated to provide a constant target negative pressure for the duration of treatment or until manually deactivated. Additionally or alternatively, the controller may have an intermittent pressure mode. For example, the controller 135 can operate the negative-pressure source 105 to cycle between a target pressure and atmospheric pressure. In some examples, the target pressure may be set at a value of 140 mmHg for a specified period of time, followed by a specified period of time of deactivation. The cycle can be repeated by activating the negative-pressure source 105, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In other examples, the target pressure can vary with time in a dynamic pressure mode. For example, the target pressure may vary in the form of a triangular waveform, varying between a negative pressure of 50 and 140 mmHg with a rise time set at a rate of +25 mmHg/min. and a descent time set at −25 mmHg/min. In other embodiments of the therapy system 100, the triangular waveform may vary between negative pressure of 25 and 140 mmHg with a rise time set at a rate of +30 mmHg/min and a descent time set at −30 mmHg/min.

In some embodiments, the controller 135 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 135, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

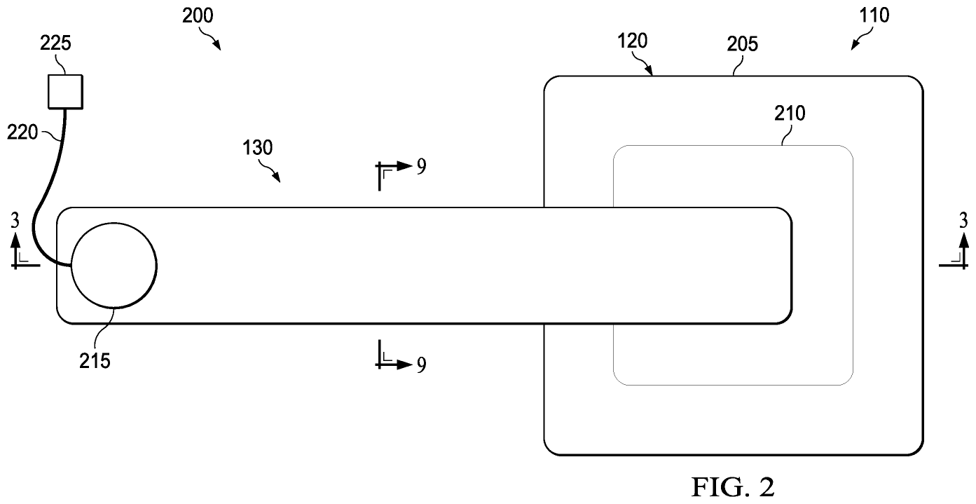

FIG. 2 is a schematic view of an example of a bridge dressing 200. As shown in the example of FIG. 2, the bridge dressing 200 may be an assembly of the dressing 110 and the fluid bridge 130. FIG. 2 illustrates additional details that may be associated with some examples of the dressing 110 and the fluid bridge 130. For example, the tissue interface 120 of FIG. 2 comprises a contact layer 205 and a fluid management layer 210. A fluid interface 215 may be disposed on the second end of the fluid bridge 130. A fluid conductor 220 with a connector 225 may optionally be connected to the fluid interface 215 in some examples. The fluid bridge 130 may be elongated to keep distribution components and other hardware away from contact points. The fluid bridge may have a length that is substantially greater than its width. For example, the fluid bridge 130 may have an aspect ratio of about 6:1 to about 12:1. A width of about two inches and a length of about 12 to 24 inches may be suitable for some embodiments. A first end of the fluid bridge 130 may be fluidly coupled to the fluid management layer 210.

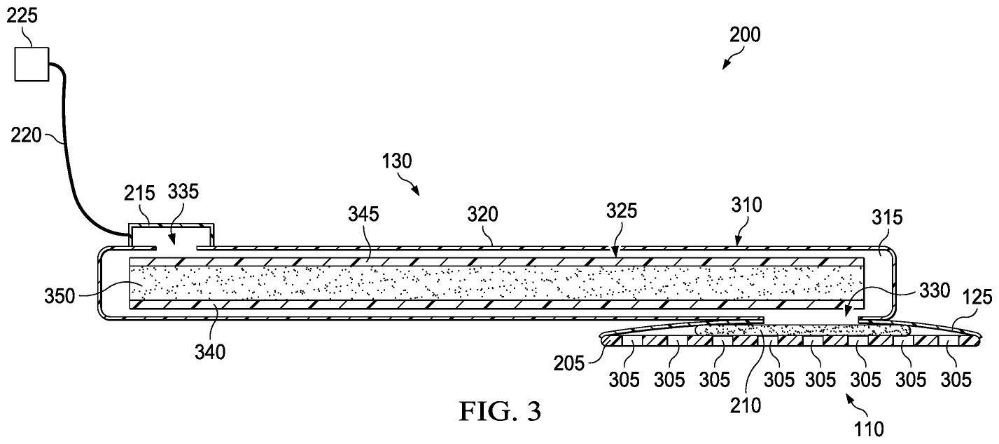

FIG. 3 is a schematic section of the bridge dressing 200 of FIG. 2, taken along line 3-3, illustrating additional details that may be associated with some embodiments. In the example configuration of FIG. 3, the contact layer 205 has apertures 305, and the fluid management layer 210 is disposed between the cover 125 and the contact layer 205. The fluid management layer 210 can separate the cover 125 and the contact layer 205, and may comprise or consist essentially of one or more fluid transfer members. In some embodiments, the fluid management layer 210 may comprise or consist essentially of a fluid transfer layer having a fluid acquisition surface and a fluid distribution surface, such as a dual-layer non-woven textile from LIBELTEX.

The fluid bridge 130 may comprise an enclosure, such as an envelope 310, which can define a fluid channel 315. The envelope 310 may be made from a material that is impermeable to liquid, and may comprise at least one vapor-transfer surface 320 that is permeable to vapor. A fluid transfer bridge 325 may be disposed within the envelope 310, adjacent to the vapor-transfer surface 320. The fluid transfer bridge 325 may be elongated, having a length that is substantially longer than its thickness and width. In some embodiments the fluid transfer bridge 325 may substantially fill the fluid channel 315 and structurally support the envelope 310. A first end of the fluid transfer bridge 325 may be fluidly coupled to the fluid management layer 210 through a first transfer channel 330. A second end of the fluid transfer bridge 325 may be fluidly coupled to a fluid interface, such as the fluid interface 215, through a second transfer channel 335. A liquid-blocking filter such as GORE MMT 314 may be disposed in, over, or between the second transfer channel 335 and the fluid interface 215 in some embodiments. The fluid transfer bridge 325 preferably has a low profile. A thickness of 15 millimeters or less may be suitable for some configurations.

The fluid transfer bridge 325 may comprise or consist essentially of one or more fluid transfer members, which may include one or more manifold members, wicking members, or some combination of manifold and wicking members. In FIG. 3, for example, the fluid transfer bridge 325 comprises a first wicking layer 340 and a second wicking layer 345. At least one fluid transfer layer may be disposed adjacent to the vapor-transfer surface 320 in some embodiments, and may be oriented to maximize adjacent surface area. For example, in FIG. 3 the second wicking layer 345 is disposed adjacent to the vapor-transfer surface 320. In some embodiments, the fluid transfer bridge 325 may comprise an intermediate fluid management member 350. For example, the fluid management member 350 may be an absorbent layer. In other examples, the fluid management member 350 may be a hydrophobic wicking layer or manifold layer. The fluid management member 350 may be adapted to distribute negative pressure between the first transfer channel 330 and the second transfer channel 335, and may also be adapted to transfer liquid between the first wicking layer 340 and the second wicking layer 345.

The thickness of fluid transfer layers in the fluid transfer bridge 325 may vary according to needs of a prescribed therapy. For example, each of the first wicking layer 340 and the second wicking layer 345 may have a thickness in a range of about 1 millimeter to about 4 millimeters. A thickness in a range of about 5 millimeters to 10 millimeters may be suitable for some embodiments of the fluid management member 350, and a thickness of about 6 millimeters may be preferable. The thickness of the fluid management member 350 may be decreased to relieve stress on other layers in some embodiments. The thickness of the fluid management member 350 can also affect the conformability of the fluid transfer bridge 325.

In some embodiments, at least a portion of the first wicking layer 340 may be in direct contact with at least a portion of the second wicking layer 345. In some embodiments, at least a portion of the first wicking layer 340 may be spaced apart or separated from the second wicking layer 345 by the fluid management member 350.

One or more of the fluid transfer layers of the fluid transfer bridge 325 may have a fluid acquisition surface and a fluid distribution surface. For example, the first wicking layer 340 may have a fluid distribution surface in contact with the fluid management member 350 and a fluid acquisition surface oriented toward the first transfer channel 330. The second wicking layer 345 may have a fluid acquisition surface in contact with the fluid management member 350 and a fluid distribution surface adjacent to or in contact with the vapor-transfer surface 320.

Additionally or alternatively, the fluid conductor 220 may be hydrophilic and evaporative. The fluid conductor 220 may further comprise hydrophilic polyurethane. The hydrophilic material can facilitate fluid absorption, and the absorbed fluid can migrate from a high-concentration state inside the fluid conductor 220 to a low-concentration state outside the fluid conductor 220 through an osmotic process. The evaporative effect may be increased by increasing the surface area of the fluid conductor 220. For example, a flat tube may be advantageous for some applications. Fins or other structures may also increase the surface area while retaining a slightly smaller inner bore. To maintain a constant wall thickness, the fluid conductor 220 may be extruded with a star-like cross-section, which can increase the surface area exposed to atmosphere and to fluid.

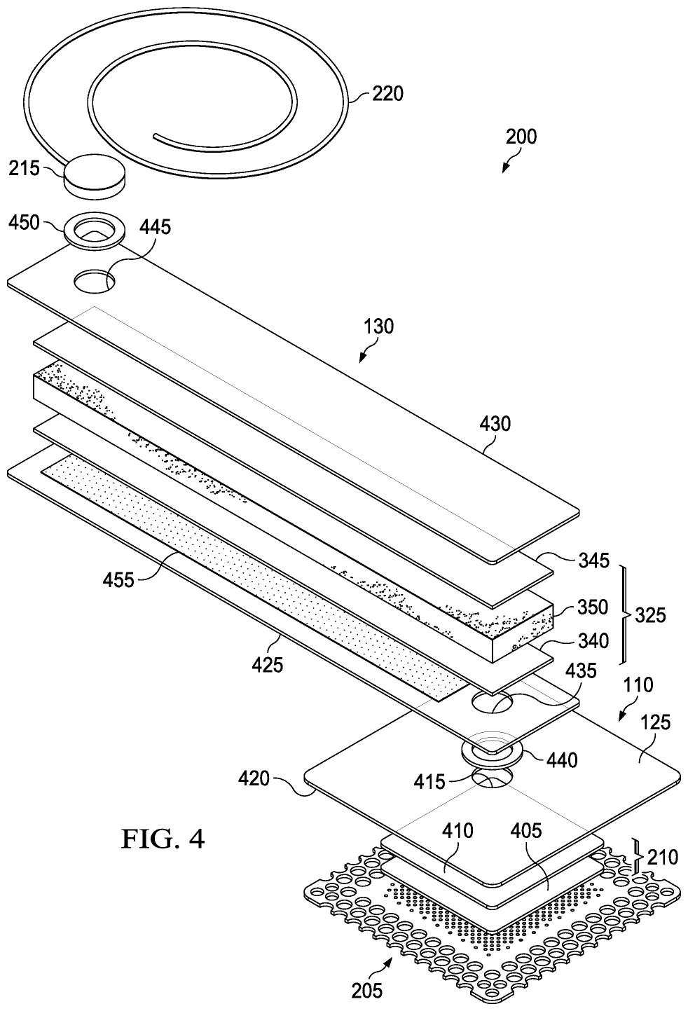

FIG. 4 is an assembly view of an example of the bridge dressing 200 of FIG. 2, illustrating additional details that may be associated with some embodiments. As illustrated in the example of FIG. 4, some embodiments of the contact layer 205 may be perforated. In some embodiments, the contact layer 205 may comprise or consist essentially of a soft, pliable material suitable for providing a fluid seal around a tissue site, and may have a substantially flat surface. For example, the contact layer 205 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. In some embodiments, the contact layer 205 may have a thickness between about 200 microns (μm) and about 1000 microns (μm). In some embodiments, the contact layer 205 may have a hardness between about 5 Shore OO and about 80 Shore OO.

In some embodiments, the contact layer 205 may be a hydrophobic-coated material. For example, the contact layer 205 may be formed by coating a spaced material, such as woven, non-woven, molded, or extruded mesh, with a hydrophobic material. The hydrophobic material for the coating may be a soft silicone, for example.

As illustrated in the example of FIG. 4, the fluid management layer 210 may comprise or consist essentially of one or more fluid transfer members, such as a third wicking layer 405 and a fourth wicking layer 410. In some examples, the third wicking layer 405 and the fourth wicking layer 410 may be disposed between the cover 125 and the contact layer 205 in a stacked relationship as shown in FIG. 4. In some examples, two or more fluid transfer layers may be laminated. For example, an adhesive or thermal weld can bond or otherwise secure the third wicking layer 405 and the fourth wicking layer 410 to each other without adversely affecting fluid transfer.

In the example of FIG. 4, the third wicking layer 405 may have a fluid acquisition surface oriented toward the contact layer 205, and the fourth wicking layer 410 may have a fluid distribution surface oriented toward the cover 125. LIBELTEX TDL2 having a weight of 80 g.s.m. or similar materials may be suitable for use as or in the third wicking layer 405, the fourth wicking layer 410, or both.

In some examples, the third wicking layer 405 may have a wider base and a higher density relative to the fourth wicking layer 410. The third wicking layer 405 may have a surface area that is greater than a surface area of the fourth wicking layer 410. The third wicking layer 405 may be thicker than the fourth wicking layer 410 in some examples. For example, the third wicking layer 405 may have a thickness of about 50 millimeters, and the fourth wicking layer 410 may have a thickness of about 20 millimeters. The third wicking layer 405 may include a profile configured to spread fluid out over an entire surface of the third wicking layer 405 to increase evaporation. The fourth wicking layer 410 may be used to pull fluid away from the third wicking layer 405. In some embodiments, the fourth wicking layer 410 may alternatively or additionally include a profile like the profile of the third wicking layer 405 to spread fluid out over an entire surface of the fourth wicking layer 410. The profile of the fourth wicking layer 410 may also be used to increase evaporation.

In some embodiments, the fluid management layer 210 may include a film between two adjacent fluid transfer layers. For example, a film may be disposed between the third wicking layer 405 and the fourth wicking layer 410. The film may include one or more of the same properties as the cover 125.

The cover 125 may be coupled to the contact layer 205 to enclose the fluid management layer 210 in some embodiments. For example, the cover 125 may be adhered to a periphery of the contact layer 205 around the fluid management layer 210. In some embodiments, the cover 125 may additionally include a fluid interface such as a first aperture 415, which may be centrally disposed over the fluid management layer 210.

The cover 125 may comprise, for example, one or more of the following materials: polyurethane (PU), such as a hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

In the example of FIG. 4, the dressing 110 may further include an attachment device, such as an adhesive 420. The adhesive 420 may be, for example, a medically-acceptable, pressure-sensitive adhesive that extends about a periphery, a portion, or the entire cover 125. In some embodiments, for example, the adhesive 420 may be an acrylic adhesive having a coating weight between 25-65 grams per square meter (g.s.m.). Thicker adhesives, or combinations of adhesives, may be applied in some embodiments to improve the seal and reduce leaks. In some embodiments, the adhesive 420 may be continuous or discontinuous layer. Discontinuities in the adhesive 420 may be provided by apertures or holes (not shown) in the adhesive 420. The apertures or holes in the adhesive 420 may be formed after application of the adhesive 420 or by coating the adhesive 420 in patterns on a carrier layer, such as, for example, a side of the cover 125. Apertures or holes in the adhesive 420 may also be sized to enhance the moisture-vapor transfer rate of the cover 125 in some example embodiments.

In some embodiments, a release liner (not shown) may be attached to or positioned adjacent to the contact layer to protect the adhesive 420 prior to use. The release liner may also provide stiffness to assist with, for example, deployment of the dressing 110. The release liner may be, for example, a casting paper, a film, or polyethylene. Further, in some embodiments, the release liner may be a polyester material such as polyethylene terephthalate (PET), or similar polar semi-crystalline polymer. The use of a polar semi-crystalline polymer for the release liner may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. In some embodiments, the release liner may have a surface texture that may be imprinted on an adjacent layer, such as the contact layer 205. Further, a release agent may be disposed on a side of the release liner that is adjacent to the contact layer 205. For example, the release agent may be a silicone coating and may have a release factor suitable to facilitate removal of the release liner by hand and without damaging or deforming the dressing 110. In some embodiments, the release agent may be a fluorocarbon or a fluorosilicone, for example. In other embodiments, the release liner may be uncoated or otherwise used without a release agent.

As illustrated in the example of FIG. 4, the fluid bridge 130 may include a base layer 425 and a top layer 430. Each of the base layer 425 and the top layer 430 may comprise or consist essentially of a material that is substantially impermeable to liquid. The base layer 425 may include a second aperture 435, which may be disposed at one end of the base layer 425 and aligned with the first aperture 415 to form a fluid interface between the dressing 110 and the fluid bridge 130. In some embodiments, for example, the first aperture 415 and the second aperture 435 may be assembled to form the first transfer channel 330 of FIG. 3. A first adhesive ring 440 may optionally be disposed around the first aperture 415, the second aperture 435, or both in some embodiments. The top layer 430 may include a third aperture 445, which may be disposed at an end opposite the second aperture 435. In some embodiments, the third aperture 445 may be aligned with an aperture (not shown) in the fluid interface 215 to form the second transfer channel 335 of FIG. 3. A second adhesive ring 450 may optionally be disposed around the third aperture 445 in some embodiments.

The base layer 425, the top layer 430, or both may comprise or consist essentially of materials similar to the cover 125. For example, the base layer 425, the top layer 430, or both may comprise or consist essentially of a vapor-transfer film. In some embodiments, suitable materials may include a film that is permeable to vapor and substantially impermeable to liquid, and may have an MVTR in a range of about 250 grams per square meter per 24 hours and about 5000 grams per square meter per 24 hours. For example, the base layer 425, the top layer 430, or both, may comprise or consist essentially of a film having an MVTR of about 2600 grams per square meter per 24 hours. Further, in some embodiments, suitable materials may be breathable. Additional examples of suitable materials may include, without limitation, a polyurethane (PU) drape or film such as SCAPA BIOFLEX 130 polyurethane film; films formed from polymers, such as polyester and co-polyester; polyamide; polyamide/block polyether; acrylics; vinyl esters; polyvinyl alcohol copolymers; and INSPIRE 2305 polyurethane drape. High-MVTR films may be advantageous for evaporation of condensate, which may occur around the entire exterior surface of the fluid bridge 130. In this manner, capacity, fluid handling, and evaporative properties of the fluid bridge 130 may be enhanced or improved due at least to increased surface area and air movement provided around all sides and portions of the exterior surface of the fluid bridge 130.

In some examples, one or more of the fluid transfer layers of the fluid transfer bridge 325 may comprise a non-woven material or structure such as, without limitation, a polyester, co-polyester, polyolefin, cellulosic fiber, and combinations or blends of these materials. In the example of FIG. 4, the first wicking layer 340, the second wicking layer 345, or both may comprise or consist essentially of a wicking textile, such as LIBELTEX TDL2 having a weight of 80 grams per square meter or similar materials. The fluid transfer layers of the fluid transfer bridge 325 preferably have a density in a range of 0.2-0.5 grams per cubic centimeter. For example, in some embodiments, the first wicking layer 340 and the second wicking layer 345 may be a textile having a density of about 0.4 grams per cubic centimeter.

In some embodiments, the fluid management member 350 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The 25% compression load deflection of the fluid management member 350 may be at least 0.35 pounds per square inch, and the 65% compression load deflection may be at least 0.43 pounds per square inch. In some embodiments, the tensile strength of the fluid management member 350 may be at least 10 pounds per square inch. The fluid management member 350 may have a tear strength of at least 2.5 pounds per inch. In some embodiments, the fluid management member 350 may comprise or consist essentially of foam polyols such as polyester or polyether, isocyanate such as toluene diisocyanate, and polymerization modifiers such as amines and tin compounds. In some examples, the fluid management member 350 may be reticulated polyurethane ether foam having a density of about 0.2 grams per cubic centimeter.

In some embodiments, the fluid management member 350 may be an absorbent. For example, the fluid management member 350 may comprise or consist essentially of a super-absorbent polymer, such as TEXSUS FP2325 or GELOK 30040-76 S/S/S absorbent, and may have a coating weight in a range of about 300 g.s.m. to about 500 g.s.m. In an unsaturated state, the fluid management member 350 may have a first volume, which can be at least 5 percent less than the internal volume of the envelope 310 and can allow for free movement of fluids and distribution of pressure around the fluid management member 350 when positioned within the envelope 310. In some embodiments, the fluid management member 350 may have an unsaturated volume that is at least 10 percent less than the internal volume of the envelope 310. In some embodiments, the fluid management member 350 may have an unsaturated volume that is between 20 percent to about 90 percent of the internal volume of the envelope 310. In some embodiments, the first wicking layer 340 and the second wicking layer 345 may entirely surround or encapsulate the fluid management member 350. Further, in some embodiments, the fluid management member 350 may be moveable, expandable, or swellable within the envelope 310. For example, the fluid management member 350 may be configured to move, expand, or swell to a second volume if the fluid management member 350 becomes fully or partially saturated.

In some embodiments, an attachment device may be disposed on an interior surface of the base layer 425, the top layer 430, or both, to secure the fluid transfer bridge 325. In some embodiments, an attachment device may be disposed between the base layer 425 and the fluid transfer bridge 325. For example, as illustrated in FIG. 4, an adhesive 455 may be coated on the interior surface of the base layer 425 to adhere the first wicking layer 340 to the base layer 425. To maximize evaporation performance of the top layer 430, there may be no adhesive between the top layer 430 and the fluid transfer bridge 325 in some embodiments.

In the example of FIG. 4, the first wicking layer 340, the fluid management member 350, and the second wicking layer 345 are stacked between the base layer 425 and the top layer 430. In some embodiments, the base layer 425 and the top layer 430 may be coupled together to form the envelope 310 of FIG. 3. For example, the base layer 425 and the top layer 430 may be sized to allow for a seal around the edges. The edges of the base layer 425 and the top layer 430 may be sealed to enclose the first wicking layer 340, the fluid management member 350, and the second wicking layer 345. The edges may be sealed by heat welding, RF welding, ultrasonic welding, or adhesives, for example.

FIG. 4 also illustrates one example of the fluid interface 215 and the fluid conductor 220. As shown in the example of FIG. 4, the fluid conductor 220 may be a flexible tube, which can be fluidly coupled on one end to the fluid interface 215. The fluid interface 215 may be an elbow connector, as shown in the example of FIG. 4, which can be placed over the third aperture 445 to provide a fluid path between the fluid conductor 220 and the fluid transfer bridge 325. The fluid interface 215 may comprise or consist essentially of a soft, medical-grade polymer or other pliable material. Examples of suitable materials include polyurethane, polyethylene, polyvinyl chloride (PVC), fluorosilicone, or ethylene-propylene. In some illustrative, non-limiting embodiments, the fluid interface 215 may be molded from DEHP-free PVC. The fluid interface 215 may be formed in any suitable manner such as by molding, casting, machining, or extruding.

In some embodiments, the fluid interface 215 may be formed of a material having absorbent properties, evaporative properties, or both. The material may be vapor permeable and liquid impermeable, which can permit vapor to be absorbed into and evaporated from the material through permeation while inhibiting permeation of liquids. The absorbent material may be, for example, a hydrophilic polymer such as hydrophilic polyurethane.

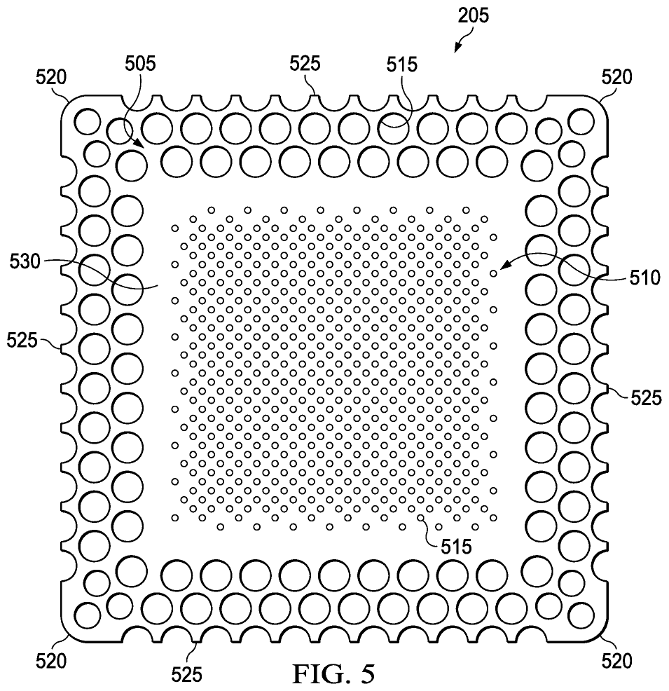

FIG. 5 is a plan view of the contact layer 205 of FIG. 4, illustrating additional details that may be associated with some embodiments. For example, the contact layer 205 may have a periphery 505 surrounding or around an interior portion 510, and may have apertures 515 disposed through the periphery 505 and the interior portion 510. The interior portion 510 may correspond to a surface area of the cover 125 in some examples. The contact layer 205 may also have corners 520 and edges 525. The corners 520 and the edges 525 may be part of the periphery 505. The contact layer 205 may have an interior border 530 around the interior portion 510, disposed between the interior portion 510 and the periphery 505. The interior border 530 may be substantially free of the apertures 515, as illustrated in the example of FIG. 5. In some examples, as illustrated in FIG. 5, the interior portion 510 may be symmetrical and centrally disposed in the contact layer 205.

The apertures 515 may be formed by cutting or by application of local RF or ultrasonic energy, for example, or by other suitable techniques for forming an opening. The apertures 515 may have a uniform distribution pattern, or may be randomly distributed on the contact layer 205. The apertures 515 in the contact layer 205 may have many shapes, including circles, squares, stars, ovals, polygons, slits, complex curves, rectilinear shapes, triangles, for example, or may have some combination of such shapes.

Each of the apertures 515 may have uniform or similar geometric properties. For example, in some embodiments, each of the apertures 515 may be circular apertures, having substantially the same diameter. In some embodiments, the diameter of each of the apertures 515 may be about 1 millimeter to about 50 millimeters. In other embodiments, the diameter of each of the apertures 515 may be about 1 millimeter to about 20 millimeters.

In other embodiments, geometric properties of the apertures 515 may vary. For example, the diameter of the apertures 515 may vary depending on the position of the apertures 515 in the contact layer 205, as illustrated in FIG. 5. In some embodiments, the diameter of the apertures 515 in the periphery 505 of the contact layer 205 may be larger than the diameter of the apertures 515 in the interior portion 510 of the contact layer 205. For example, in some embodiments, the apertures 515 disposed in the periphery 505 may have a diameter between about 9.8 millimeters to about 10.2 millimeters. In some embodiments, the apertures 515 disposed in the corners 520 may have a diameter between about 7.75 millimeters to about 8.75 millimeters. In some embodiments, the apertures 515 disposed in the interior portion 510 may have a diameter between about 1.8 millimeters to about 2.2 millimeters.

At least one of the apertures 515 in the periphery 505 of the contact layer 205 may be positioned at the edges 525 of the periphery 505, and may have an interior cut open or exposed at the edges 525 that is in fluid communication in a lateral direction with the edges 525. The lateral direction may refer to a direction toward the edges 525 and in the same plane as the contact layer 205. As shown in the example of FIG. 5, the apertures 515 in the periphery 505 may be positioned proximate to or at the edges 525 and in fluid communication in a lateral direction with the edges 525. The apertures 515 positioned proximate to or at the edges 525 may be spaced substantially equidistant around the periphery 505 as shown in the example of FIG. 5. Alternatively, the spacing of the apertures 515 proximate to or at the edges 525 may be irregular.

Various components of the bridge dressing 200 may be assembled before application or in situ. For example, the cover 125 may be laminated to the fluid management layer 210, and the fluid management layer 210 may be laminated to the contact layer 205 opposite the cover 125 in some embodiments. In some embodiments, one or more layers of the dressing 110 may be coextensive. For example, the contact layer 205 may be coextensive with the cover 125, as illustrated in the example of FIG. 4. In some embodiments, the dressing 110 may be provided as a single, composite dressing. For example, the contact layer 205 may be coupled to the cover 125 to enclose the fluid management layer 210, wherein the contact layer 205 is configured to face a tissue site. Additionally or alternatively, the fluid bridge 130 may be provided as a composite structure, and may be provided attached or unattached to the dressing 110.

In use, the release liner (if included) may be removed to expose the contact layer 205, which may be placed within, over, on, or otherwise proximate to a tissue site. The contact layer 205 may be sufficiently tacky to hold the dressing 110 in position, while also allowing the dressing 110 to be removed or re-positioned without trauma to a tissue site.

Removing the release liner can also expose adhesive, such as the adhesive 420, and the cover 125 may be attached to an attachment surface. For example, the cover 125 may be attached to epidermis peripheral to a tissue site, around the fluid management layer 210. In the example of FIG. 5, the adhesive 420 may be in fluid communication with an attachment surface through the apertures 515 in at least the periphery 505 of the contact layer 205. The adhesive 420 may also be in fluid communication with the edges 525 through the apertures 515 exposed at the edges 525.

Once the dressing 110 is in a desired position, the adhesive 420 may be pressed through the apertures 515 to bond the dressing 110 to the attachment surface. The apertures 515 at the edges 525 may permit the adhesive 420 to flow around the edges 525 for enhancing the adhesion of the edges 525 to an attachment surface.

In some embodiments, apertures 515 in the contact layer 205 may be sized to control the amount of the adhesive 420 in fluid communication with an attachment surface through the apertures 515. For a given geometry of the corners 520, the relative sizes of the apertures 515 may be configured to maximize the surface area of the adhesive 420 exposed and in fluid communication through the apertures 515 at the corners 520. For example, as shown in FIG. 5, the edges 525 may intersect at a substantially right angle, or about 90 degrees, to define the corners 520. In some embodiments, the corners 520 may have a radius of about 10 millimeters. Further, in some embodiments, three of the apertures 515 having a diameter between about 7.75 millimeters to about 8.75 millimeters may be positioned in a triangular configuration at the corners 520 to maximize the exposed surface area for the adhesive 420. In other embodiments, the size and number of the apertures 515 in the corners 520 may be adjusted as necessary, depending on the chosen geometry of the corners 520, to maximize the exposed surface area of the adhesive 420. Further, the apertures 515 at the corners 520 may be fully housed within the contact layer 205, substantially precluding fluid communication in a lateral direction exterior to the corners 520. The apertures 515 at the corners 520 being fully housed within the contact layer 205 may substantially preclude fluid communication of the adhesive 420 exterior to the corners 520, and may provide improved handling of the dressing 110 during deployment at a tissue site. Further, the exterior of the corners 520 being substantially free of the adhesive 420 may increase the flexibility of the corners 520 to enhance comfort.

In some embodiments, the bond strength of the adhesive 420 may vary in different locations of the dressing 110. For example, the adhesive 420 may have lower bond strength in locations adjacent to the contact layer 205 where the apertures 515 are relatively larger, and may have higher bond strength where the apertures 515 are smaller. Adhesive 420 with lower bond strength in combination with larger apertures 515 may provide a bond comparable to adhesive 420 with higher bond strength in locations having smaller apertures 515.

The fluid bridge 130 may be fluidly coupled to the dressing 110, if appropriate, and the fluid bridge 130 may be fluidly coupled to the negative-pressure source 105. In some embodiments, the fluid bridge 130 may be coupled to the negative-pressure source 105 through the fluid interface 215. For example, if not already configured, the fluid interface 215 may be disposed over the third aperture 445 and attached to the fluid bridge 130. The fluid conductor 220 may be fluidly coupled to the fluid interface 215 and to the negative-pressure source 105.

In some examples, the fluid bridge 130 may be secured with skin-friendly adhesive pads along the length of the fluid bridge 130. Suitable materials may include silicone or polyurethane gels, for example.

Thus, the dressing 110 can provide a sealed therapeutic environment proximate to a tissue site, substantially isolated from the external environment, and the negative-pressure source 105 can reduce the pressure in the sealed therapeutic environment. The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

The contact layer 205 may provide an effective and reliable seal against challenging anatomical surfaces, such as an elbow or heel, at and around a tissue site. Further, the dressing 110 may permit re-application or re-positioning, to correct air leaks caused by creases and other discontinuities between the dressing 110 and a tissue site. The ability to rectify leaks may increase the efficacy of the therapy and reduce power consumption in some embodiments.

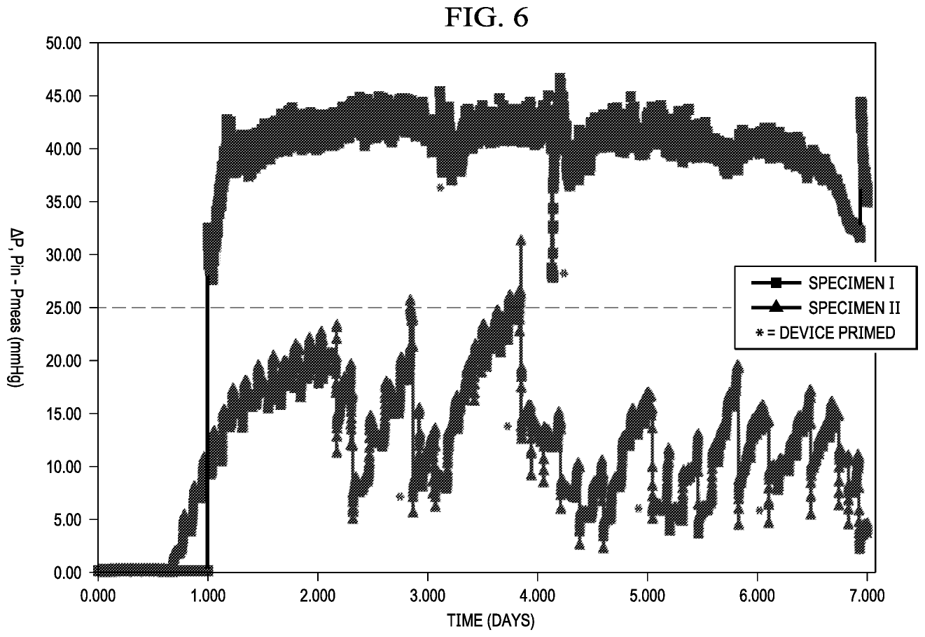

The bridge dressing 200 can also minimize pressure drops between a negative-pressure source and a tissue site. FIG. 6 is illustrative of pressure drop performance that may be associated with some features of the bridge dressing 200. FIG. 6 represents pressure data collected over a treatment period of 7 days from two specimens. Specimen I included a dressing coupled to a manual pump with integrated fluid storage. A tube having a length of 510 millimeters was used to fluidly couple the dressing of Specimen I to the manual pump. Specimen I did not have a bridge. The average pressure differential between the pump pressure and test site for Specimen I was 38.4 mmHg. Specimen II included a dressing with an evaporative bridge fluidly coupled to the same type of manual pump with integrated fluid storage. The evaporative bridge of Specimen II also had a length of 510 millimeters. The average pressure differential between the pump pressure and test site for Specimen II was 11.9 mmHg. As evidenced by FIG. 6, Specimen II maintained a pressure drop over the period that was generally less than 25 mmHg, or about 0.05 mmHg per millimeter between the pump and the tissue site, substantially less than the pressure drop maintained by Specimen I.

In some embodiments, couplings may be used to facilitate replacement of the dressing 110, the fluid bridge 130, or other components.

In some applications, a filler may also be disposed between a tissue site and the contact layer 205. For example, if the tissue site is a surface wound, a wound filler may be applied interior to the periwound, and the contact layer 205 may be disposed over the periwound and the wound filler. In some embodiments, the filler may be a manifold, such as open-cell foam.

Additionally or alternatively, compression may be applied to portions of the bridge dressing 200 in some treatment applications. For example, the bridge dressing 200 may be placed on a wound, and breathable bandages or compression garments may be placed over at least portions of the bridge dressing 200 in some embodiments.

Negative pressure applied through the dressing 110 across a tissue site in a sealed therapeutic environment can induce macrostrain and micro-strain in the tissue site, as well as remove exudates and other fluids from the tissue site, which can be collected in the container 115. The fluid management layer 210 can preference exudate and other fluid away from a tissue site and substantially prevent fluid from returning to the tissue site.

As exudate is drawn into the fluid bridge 130, the relative humidity in the fluid bridge 130 may increase. In some examples, the relative humidity may increase to 100%, either locally or across the entire length of the fluid bridge 130. If the ambient relative humidity is less than the relative humidity in the fluid bridge 130, a humidity gradient across the vapor-transfer surface 320 can cause vapor to egress the fluid bridge 130 through the vapor-transfer surface 320. Vapor may be transferred across the vapor-transfer surface 320 until reaching humidity equilibrium across the vapor-transfer surface 320. In some examples, the vapor-transfer surface 320 may be textured or pleated to increase the surface area available for transfer. The humidity gradient may be maintained by moving drier air across the vapor-transfer surface 320.

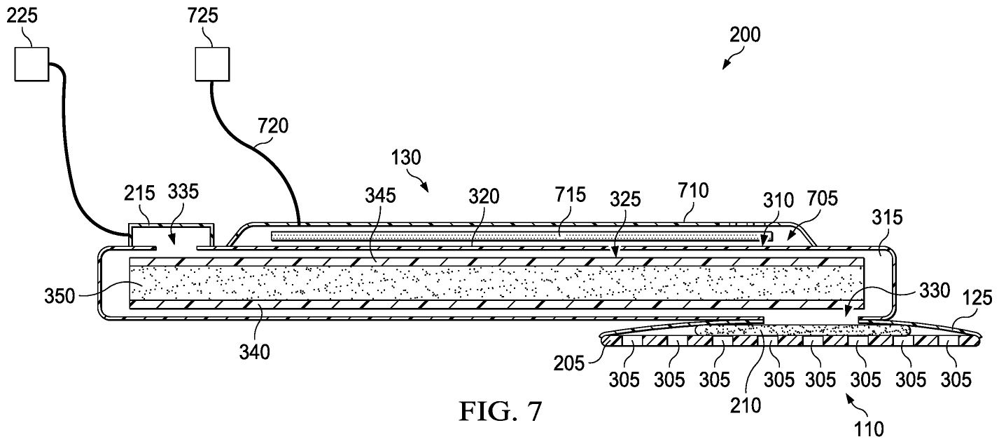

FIG. 7 is a schematic section of another example of the bridge dressing 200 of FIG. 2, taken along line 3-3, illustrating additional details that may be associated with some embodiments. In the example of FIG. 7, the bridge dressing 200 includes an evaporation channel 705 disposed adjacent to the vapor-transfer surface 320. In some embodiments, the evaporation channel 705 may be defined by an additional layer of material that is substantially impermeable to liquid, which can be coupled to the envelope 310. In other embodiments, the evaporation channel 705 may be defined by a pouch or envelope coupled to the envelope 310. In operation, controlled air-flow through the evaporation channel 705 can maintain the humidity gradient, with moist air being exhausted to the local environment.

In the example of FIG. 7, the evaporation channel 705 is defined by a cover 710, which may be coupled to the envelope 310 over at least a portion of the vapor-transfer surface 320. The cover 710 may comprise or consist essentially of a material that is the same or similar to the material of the cover 125. The cover 710 may comprise or consist essentially of a vapor-transfer film. In some embodiments, suitable materials may include a film that is permeable to vapor and substantially impermeable to liquid, and may have an MVTR in a range of about 250 grams per square meter per 24 hours and about 5000 grams per square meter per 24 hours. For example, cover 710 may comprise or consist essentially of a film having an MVTR of about 2600 grams per square meter per 24 hours. Further, in some embodiments, suitable materials may be breathable. Additional examples of suitable materials may include, without limitation, a polyurethane (PU) drape or film such as SCAPA BIOFLEX 130 polyurethane film; films formed from polymers, such as polyester and co-polyester; polyamide; polyamide/block polyether; acrylics; vinyl esters; polyvinyl alcohol copolymers; and INSPIRE 2304 polyurethane drape.

The evaporation channel 705 may also include a support means to keep the evaporation channel 705 open under external or internal pressure. For example, a filler medium may be disposed within the evaporation channel 705 to provide support. In some embodiments, one or more fluid transfer members may be a suitable filler medium. The fluid transfer member may include one or more manifold members, wicking members, or some combination of manifold and wicking members. In FIG. 7, for example, an evaporation manifold 715 is disposed in the evaporation channel 705. In some examples, the evaporation manifold 715 may comprise a non-woven material or structure such as, without limitation, a polyester, co-polyester, polyolefin, cellulosic fiber, and combinations or blends of these materials. In some embodiments, the evaporation manifold 715 may comprise or consist essentially of a wicking textile, such as LIBELTEX TDL4 having a weight of 150 g.s.m. or similar materials. Foams or 3D spacer fabrics from manufacturers such as Baltex may also be suitable for some embodiments.

A fluid conductor 720 with a connector 725 may optionally be connected to the evaporation channel 705 in some examples.

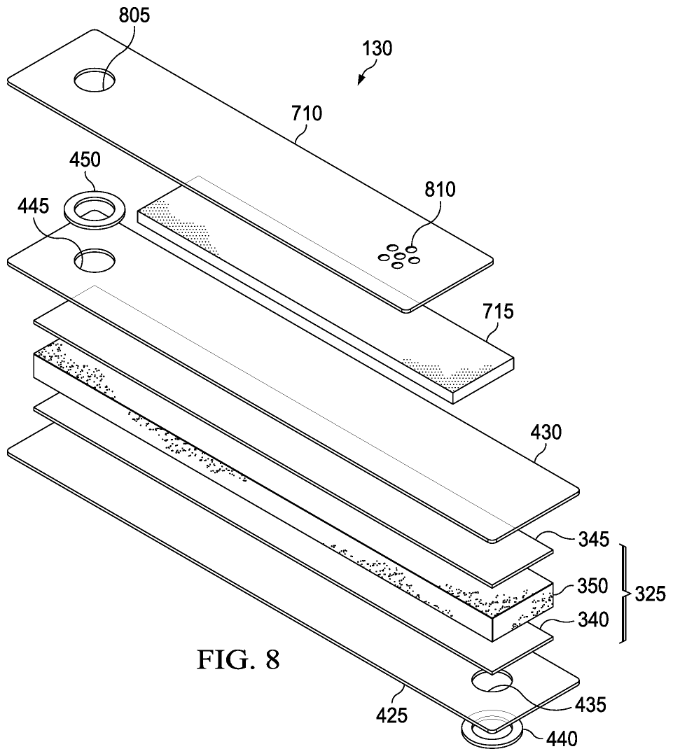

FIG. 8 is an assembly view of an example of the fluid bridge 130 of FIG. 2, illustrating additional details that may be associated with some embodiments. In the example of FIG. 8, the first wicking layer 340, the fluid management member 350, and the second wicking layer 345 are stacked between the base layer 425 and the top layer 430. In some embodiments, the base layer 425 and the top layer 430 may be coupled together to form the envelope 310 of FIG. 7. For example, the edges of the base layer 425 and the top layer 430 may be welded together to enclose the first wicking layer 340, the fluid management member 350, and the second wicking layer 345. The evaporation manifold 715 may be disposed between the top layer 430 and the cover 710, and the edges of the cover 710 may be coupled to the top layer 430 around the evaporation manifold 715. In the example of FIG. 8, the cover 710 has first fluid interface 805 and a second fluid interface 810. The first fluid interface 805 and the second fluid interface 810 may be at opposing ends of the cover 710. In use, the first fluid interface 805 and the second fluid interface 810 may allow the flow of air and water vapor through evaporation channel 705.

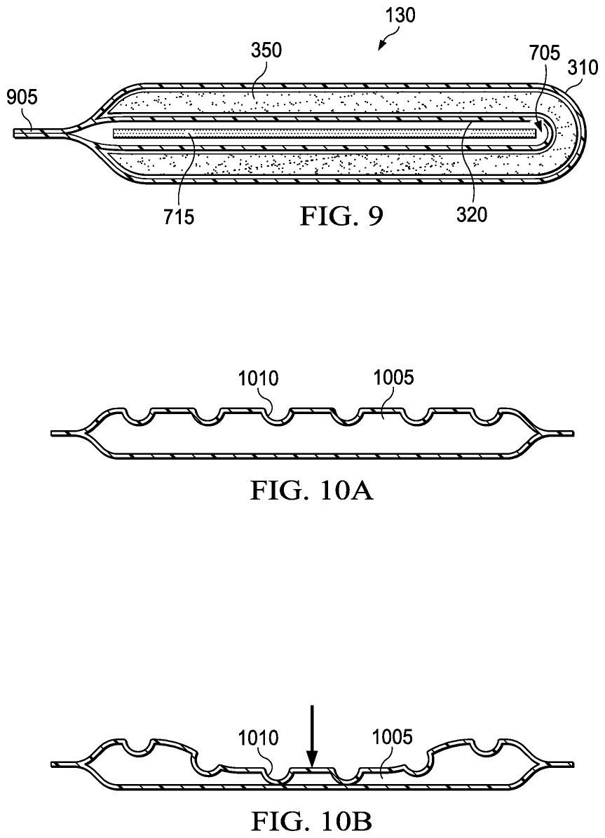

FIG. 9 is a schematic section of another example of the fluid bridge 130 of FIG. 2, taken along line 9-9, illustrating additional details that may be associated with some embodiments. In some embodiments, the evaporation channel 705 may be formed by folding one or more of the fluid transfer members over on itself along its length and sealing along the open edge. In the example of FIG. 9, the evaporation channel 705 is formed by folding the envelope 310 over the evaporation manifold 715 so that the vapor-transfer surface 320 is adjacent to the evaporation channel 705, and sealing along an edge 905.

FIG. 10A and FIG. 10B are schematic diagrams illustrating other features that may be associated with some embodiments of the fluid bridge 130 of FIG. 1. In some embodiments, the fluid bridge 130 may not include wicking or fluid transport materials within one or more pathways within the bridge. However, in any embodiment, the fluid bridge 130 may include features to reduce the chance that a passageway within the fluid bridge 130 might be pinched or occluded. The walls of the fluid bridge may also include features to assist with fluid evaporation in addition to those discussed above.

FIG. 10A, for example, illustrates a support means that may be associated with a flow channel, such as the evaporation channel 705. As illustrated in FIG. 10A, the support means may comprise a textured surface on at least one side of a flow channel 1005. In some embodiments, a suitable textured surface may comprise a raised pattern of protrusions, such as bosses 1010. The pattern may be a regular pattern of repeating features and intervals between features. The bosses 1010 may be formed by embossing a film that defines the flow channel 1005. For example, the cover 710 may be vacuum-formed to create bosses in the evaporation channel 705. The shape of the bosses 1010 may vary. As illustrated in FIG. 10A, the bosses 1010 may have a semi-circular profile in some examples. Semi-hemispherical bubbles or blisters may be particularly suitable for some embodiments. In some embodiments, the bosses 1010 may have a width of about 2.5 millimeters and a height of about 1 millimeter. Dimensions of the bosses 1010 may vary, and a suitable range for the width may be about 2-3 millimeters and the height may be about 0.5-1.5 millimeters. External pressure applied to the flow channel 1005 may cause some portion of the flow channel 1005 to collapse, but the bosses 1010 can provide recessed channels along a surface of the flow channel 1005 to maintain an open flow path as illustrated in FIG. 10B. For example, recessed channels formed by the bosses 1010 can provide a pathway through the flow channel 1005 if external pressure partly blocks the flow channel 1005. The bosses 1010 may be used in addition to or instead of other support means, such as a filler medium.

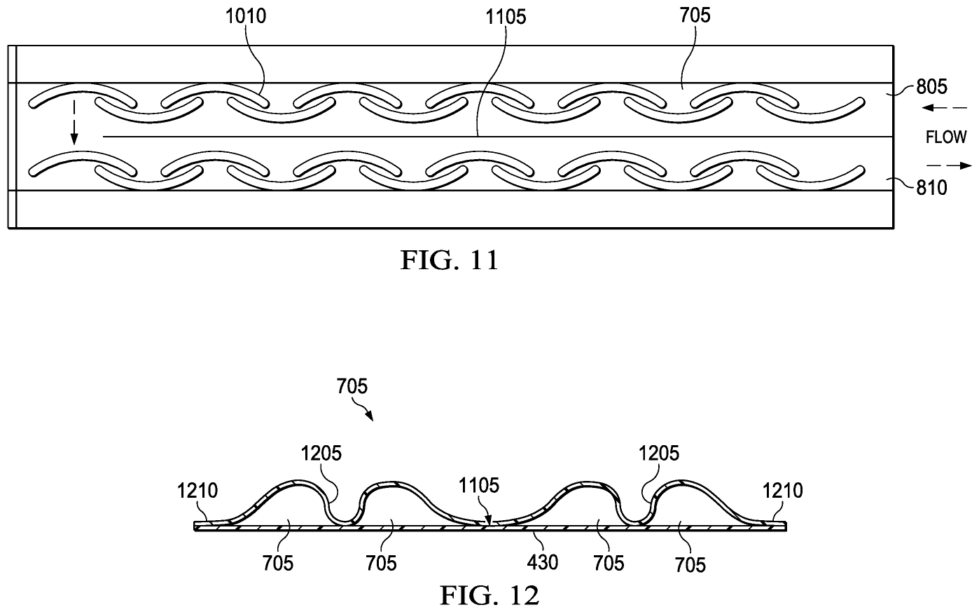

FIG. 11 is a schematic diagram of another example configuration of the evaporation channel 705. In the example of FIG. 11, the evaporation channel 705 has a tortuous path, including a return path. In some embodiments, the tortuous path may be defined at least in part by a baffle 1105 in an interior portion of the evaporation channel 705. In some embodiments, the baffle 1105 may be coupled to one or more surfaces of the evaporation channel 705, or may be formed by adjacent walls of parallel channels. In other embodiments, the baffle 1105 may be formed by welding two surfaces of the evaporation channel 705. For example, a center portion of the cover 710 may be welded to the top layer 430 in some embodiments. The tortuous path may be further defined by bosses 1010. In the example of FIG. 11, the bosses 1010 may be arced along a length of the evaporation channel 705, which may increase turbulence and improve evaporation. Bosses having an arcuate shape, such as in the bosses 1010 of FIG. 11, may have a width of about 2 to 3 millimeters and a height of about 1.5 to 2 millimeters in some embodiments. In some embodiments, the first fluid interface 805 and the second fluid interface 810 may be at the same end of the cover 710 and may be in fluid communication with the return path, as illustrated in FIG. 11.

FIG. 12 is a schematic section of the evaporation channel 705 of FIG. 11, illustrating additional details that may be associated with some embodiments. For example, FIG. 12 illustrates embossed corrugations 1205 as another means for supporting the evaporation channel 705. Edges 1210 of the cover 710 may be bonded to the top layer 430 or other film along at least a portion of the length of the evaporation channel 705. FIG. 12 also illustrates an example configuration of the baffle 1105, in which a center portion of the cover 710 is bonded to the top layer 430 or other film. Bonding the edges 1210 and the center portion of the cover 710 may also provide a means for supporting the evaporation channel 705, in addition to or instead of the embossed corrugations 1205.

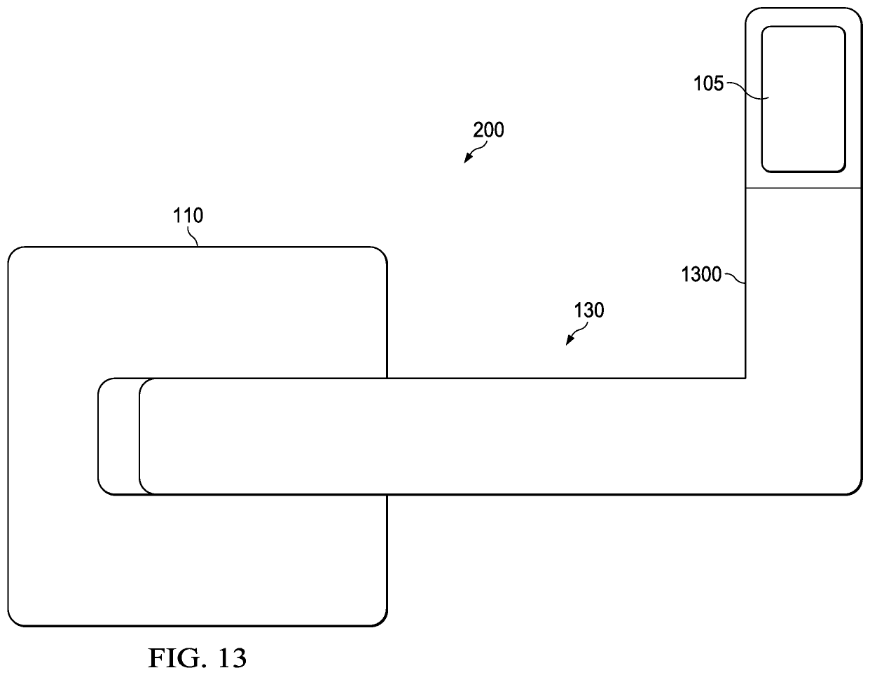

FIG. 13 illustrates another example configuration of the bridge dressing 200 having an arm or other extension that can allow access to tissue sites that may be difficult to reach. In the example of FIG. 13, the bridge dressing 200 comprises an arm 1300 that fluidly couples the negative pressure source 105 to the fluid bridge 130 and forms a substantially right angle with the fluid bridge 130. In some embodiments, the arm 1300 may have substantially the same structure as the fluid bridge 130. The L-shaped configuration of FIG. 13 may be particularly advantageous for application to a sacral pressure ulcer. For example, the dressing 110 and the fluid bridge 130 may be aligned with the spine, and the arm 1300 may be disposed around the torso to a free space at the side of a patient without raising a contact pressure point. The right angle is merely illustrative, though, and other angles may be suitable or preferable for certain tissue sites.

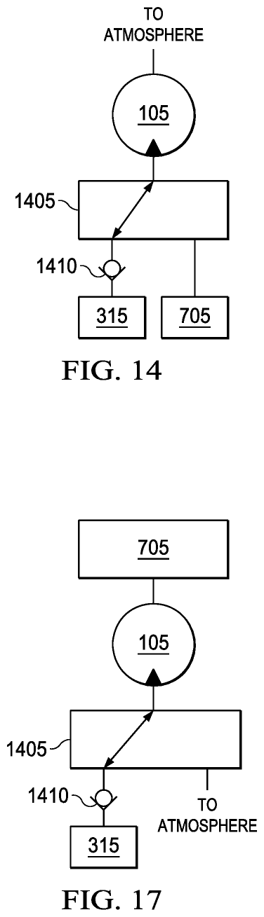

FIG. 14 is a functional block diagram illustrating additional details that may be associated with some embodiments of the therapy system 100. In the example of FIG. 14, the therapy system 100 comprises a flow controller, such as an air-source selection valve 1405, which may alternately couple the negative-pressure source 105 to the fluid bridge 130 through fluid channel 315 or the evaporation channel 705. In the state illustrated in FIG. 14, the air-source selection valve 1405 fluidly couples the negative-pressure source 105 to the fluid channel 315. The fluid channel 315 may be fluidly coupled to the air-source selection valve 1405 through a one-way valve 1410. The one-way valve 1410 allows fluid, such as air and exudate, to flow through the one-way valve 1410 from fluid channel 315 toward air-source selection valve 1405, but prevents fluid flow in the opposite direction. The air-source selection valve 1405 may be controlled by the controller 135 in some embodiments. Additionally or alternatively, the air-source selection valve 1405 may be configured for manual actuation.

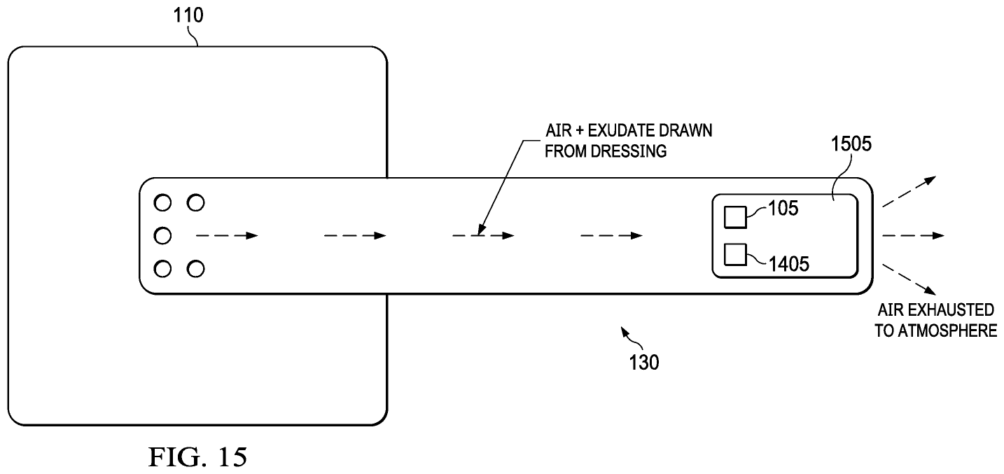

FIG. 15 is a schematic diagram of an example of the therapy system 100 that illustrates additional details that may be associated with the operation of the air-source selection valve 1405 of FIG. 14. In the example of FIG. 15, the negative-pressure source 105 and the air-source selection valve 1405 may be combined into a therapy unit 1505. In operation, the negative-pressure source 105 can pull fluid, such as air and exudate, through the dressing 110 and the fluid channel 315, and pressurized air from the negative-pressure source 105 can be exhausted to atmosphere. The negative-pressure source 105 may be fluidly coupled to the fluid channel 315 opposite the dressing 110 to maximize transfer through the fluid channel 315 in some examples. A valve may be used to draw low-humidity air into the fluid channel 315 from atmosphere.

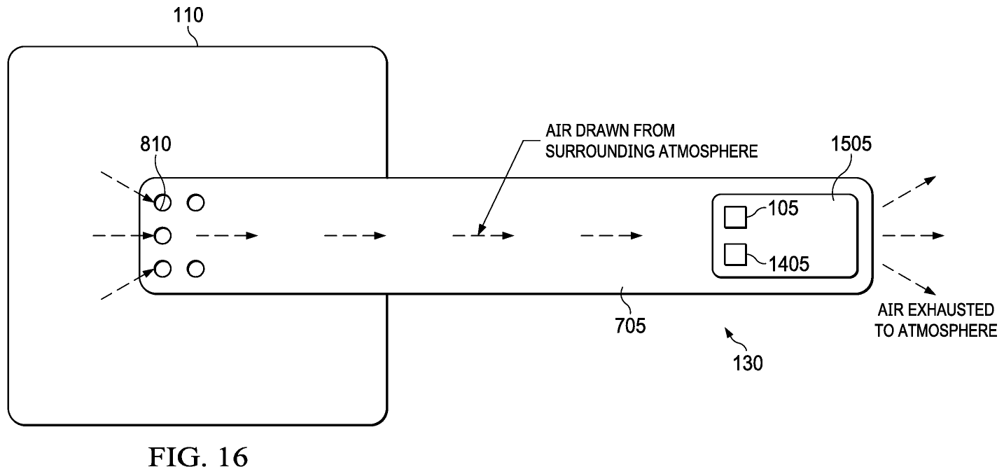

FIG. 16 is a schematic diagram of the therapy system 100 of FIG. 15, illustrating additional details that may be associated with the operation if the air-source selection valve 1405 is switched to fluidly couple the negative-pressure source 105 to the evaporation channel 705. As illustrated in FIG. 16, the negative-pressure source 105 can pull air from the second fluid interface 810, through the evaporation channel 705, and exhaust the air to atmosphere.

FIG. 17 is a schematic diagram illustrating additional details that may be associated with some embodiments of the therapy system 100. In the example of FIG. 17, the evaporation channel 705 is fluidly coupled to the exhaust (positive pressure) side of the negative-pressure source 105, and the air-source selection valve 1405 may alternately couple the negative-pressure source 105 to the fluid channel 315 or atmosphere. In the state illustrated in FIG. 17, the air-source selection valve 1405 fluidly couples the negative-pressure source 105 to the fluid channel 315.

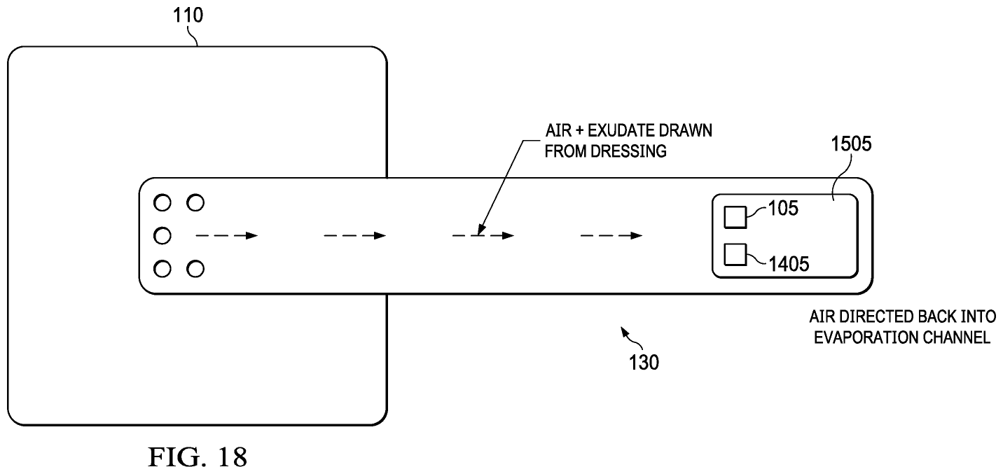

FIG. 18 is a schematic diagram of an example of the therapy system 100 that illustrates additional details that may be associated with the operation of the air-source selection valve 1405 of FIG. 17. In operation, the negative-pressure source 105 can pull fluid, such as air and exudate, through the dressing 110 and the fluid channel 315, and pressurized air from the negative-pressure source 105 can be exhausted through the evaporation channel 705 and the second fluid interface 810 to atmosphere.

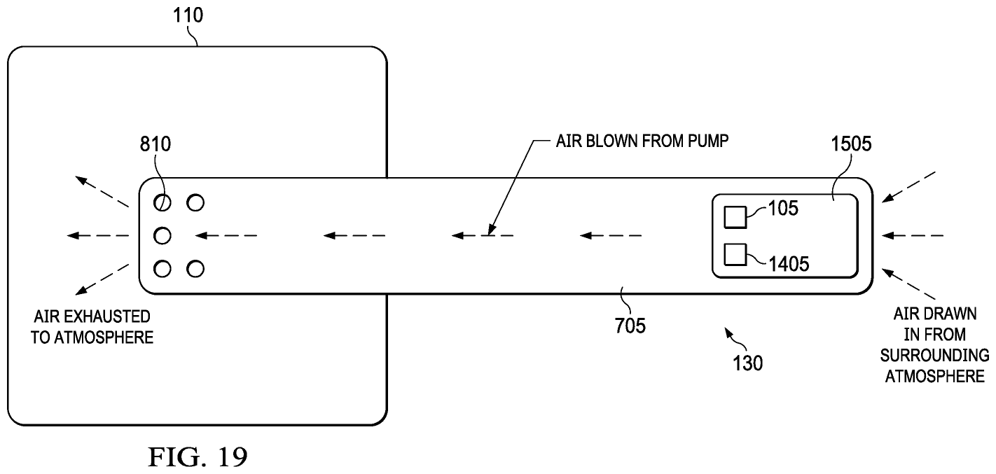

FIG. 19 is a schematic diagram of the therapy system 100 of FIG. 18, illustrating additional details that may be associated with the operation if the air-source selection valve 1405 is switched to fluidly couple the negative-pressure source 105 to atmosphere. As illustrated in FIG. 19, the negative-pressure source 105 can pull air from atmosphere, exhausting the air through the evaporation channel 705 and the second fluid interface 810.

Additionally or alternatively, the therapy system 100 may have a separate positive-pressure source, such as a blower or fan, to provide evaporative flow. Fans or blowers may operate at a lower noise level than a negative-pressure source such as a vacuum pump. Suitable piezoelectric blowers may operate at supersonic frequencies. Murata manufactures such a blower that is small and can provide 2.5 liters of air per minute. The controller 135 may operate the positive-pressure source independently or concurrently with the negative-pressure source in some examples. An air-source selection valve may also be used to switch between sources in some examples.

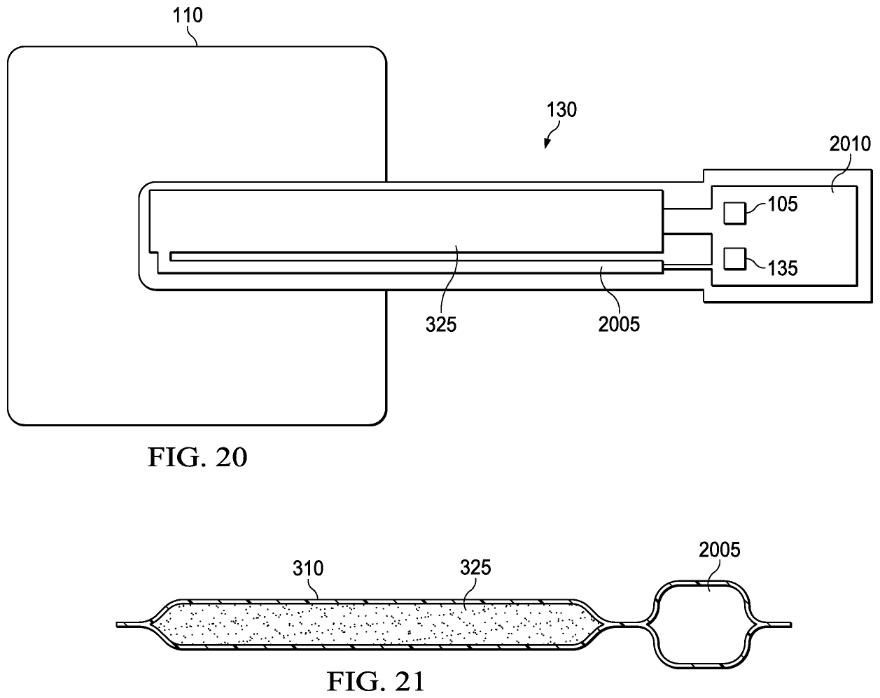

FIG. 20 is a schematic view of another example of the therapy system 100, illustrating additional details that may be associated with some embodiments. For example, the fluid bridge 130 of FIG. 20 comprises a means for measuring pressure adjacent to the second aperture 435. In FIG. 20, for example, a feedback path 2005 comprises a fluid conductor between two ends of the fluid bridge 130, and may be substantially parallel to the fluid transfer bridge 325. In some embodiments, the fluid conductor may be a conduit integral to the fluid bridge 130, or may be a tube attached to the fluid bridge 130, for example. In some embodiments, the feedback path 2005 may be a combination of fluid conductors. The feedback path 2005 may be fluidly coupled to a sensor associated with the controller 135, which may be combined in a therapy unit 2010 with the negative-pressure source 105 and other components in some examples. A suitable filter may be used to prevent contamination of the feedback path 2005. The feedback path 2005 may be used in combination with other features described herein, including the evaporation channel 705 and various means for supporting a flow channel. For example, the feedback path 2005 may have a textured interior surface or a filler medium to resist crushing.

FIG. 21 is a schematic section of the fluid bridge 130 of FIG. 20, illustrating additional details that may be associated with some embodiments. In the example of FIG. 21, the feedback path 2005 is integral to the fluid bridge 130. In some embodiments, the feedback path 2005 may be formed by extending the envelope 310 and sealing opposing surfaces around the feedback path 2005.

In other examples, a means for measuring pressure may comprise a remote sensor, which can transmit pressure data wirelessly to the controller 135. A wireless system may be advantageous for measuring pressure across the bridge dressing 200 instead of just at the second aperture 435, for example, which can allow pressure loss assessment to be extended further to increase accuracy. A wireless system may also be advantageous if multiple dressings are bridged.

Additionally or alternatively, some measurement and control functions may be provided with a pneumatic switch. For example, a pneumatic switch can compare pressure at a negative-pressure source with the pressure at the end of the fluid bridge 130. In some embodiments, a pneumatic switch may comprise a cavity divided into two chambers by a flexible member, such as a diaphragm. The chambers may be pneumatically isolated by the flexible member, which may be biased towards one side by an elastic element, such as a spring. An electrical switch may be formed by conductive material on the flexible member, which can complete a circuit with a pair of contacts mounted in the spring side of the chamber if it is deflected to a given position. The spring side cavity may be exposed to negative pressure at the negative-pressure source, and the other side may be connected to the pressure at the opposite end of the fluid bridge 130. If the pressure drop across the fluid bridge 130 is sufficient, the imbalance in pressure can force the flexible member against the elastic element to a point where the electrical circuit is completed. Completing the circuit can activate various functions, such as activating a positive-pressure source through the evaporation channel 705, increasing power to the negative-pressure source 105, providing an alert, or some combination of such functions. Multiple switches with different spring rates may be used to provide pressure control and graduated responses in some examples.

In use, pressures from each end of the fluid bridge 130 can be compared by the controller 135, and the controller 135 may take actions based on the comparison. For example, the controller 135 may take a first pressure sample at the negative-pressure source 105 and a second pressure sample at the second aperture 435. Based on a comparison of the first pressure sample and the second pressure sample, the controller 135 may take actions such as adjusting the negative-pressure source 105 to compensate for any pressure drop across the fluid bridge 130. Additionally or alternatively, a positive-pressure source can be activated if the pressure drop exceeds a given threshold, and deactivated if the pressure drop falls below the threshold. In an active system, the duty cycle of a positive-pressure source (e.g., a fan) may be proportional to the calculated saturation level to maintain saturation within a set band while minimizing power. The controller 135 may also activate an alarm or other indicator if saturation exceeds a given threshold. Fill rate may also be monitored and an alert provided if outside expected bounds.

The systems, apparatuses, and methods described herein may provide significant advantages. For example, some embodiments can provide a means for applying therapeutic negative pressure and to store exudate without increasing the risk of pressure-related injuries or trauma, particularly if a patient is immobile. Lower-cost, disposable components may be used in some examples. Additionally or alternatively, the time between dressing changes can be extended by managing fluid with absorption, evaporation, or both. In some examples, fluid management can also reduce pressure drop, which can be caused by saturation. Evaporation in some examples can also produce a noticeable cooling effect (approximately 5 degrees C.) that can have additional therapeutic benefits.

Some embodiments can reduce noise and fluid storage, eliminating external fluid containers and allowing the therapy system to be worn discretely. Couplings can allow some components to be re-used, and facilitate proper disposal of other components.

Additionally or alternatively, some examples may provide pressure feedback, and negative-pressure can be increased to compensate for pressure drops. Some embodiments may include active evaporation sub-systems, and a fan or blower can be powered up to a level suitable for a detected level of saturation while minimizing power consumption.

The bridge dressing 200 may be particularly beneficial for treating venous leg ulcers. A venous leg ulcer is a specialized wound that typically occurs on the lower leg, just above the ankle. An ulcer can take anywhere from four to six weeks to heal with current treatment options. Treating a venous leg ulcer with negative pressure may control exudate, encourage blood flow, and promote healing. Negative-pressure therapy may also be used in combination with compression therapy, and the bridge dressing 200 can minimize disruption of compression therapy by minimizing dressing changes.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 135 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for managing fluid from a tissue site, the apparatus comprising:
a fluid transfer bridge;
an envelope enclosing the fluid transfer bridge, the envelope comprising:
a vapor-transfer surface,
a first transfer channel, and
a second transfer channel; and
an evaporation channel disposed adjacent to the vapor-transfer surface.

2. The apparatus of claim 1, wherein the vapor-transfer surface has a moisture-vapor transfer rate of about 250 grams per square meter per twenty-four hours and about 5000 grams per square meter per twenty-four hours.

3. The apparatus of claim 1, wherein the fluid transfer bridge comprises an absorbent.

4. The apparatus of claim 1, wherein the fluid transfer bridge comprises a super-absorbent polymer.

5. The apparatus of claim 1, wherein the fluid transfer bridge comprises:
a first wicking layer;
a second wicking layer; and
an absorbent disposed between the first wicking layer and the second wicking layer.

6. The apparatus of claim 1, wherein the fluid transfer bridge comprises:
a first wicking layer having a distribution surface;
a second wicking layer having an acquisition surface; and
an absorbent disposed between the first wicking layer and the second wicking layer in contact with the distribution surface and the acquisition surface.

7. The apparatus of claim 1, wherein the fluid transfer bridge comprises:
an absorbent; and
a wicking layer having an acquisition surface in contact with the absorbent and a distribution surface adjacent to the vapor-transfer surface.

8. The apparatus of claim 1, wherein at least one of the envelope and the evaporation channel comprises a support means.

9. The apparatus of claim 1, wherein the envelope is embossed.

10. The apparatus of claim 1, wherein the envelope further comprises a textured surface configured to maintain an open flow path under external pressure.

11. The apparatus of claim 1, wherein the envelope further comprises a raised pattern of protrusions configured to support the envelope under external pressure.

12. The apparatus of claim 1, wherein the envelope further comprises recessed channels configured to maintain an open flow path under external pressure.

13. The apparatus of claim 1, wherein the envelope further comprises bosses configured to support the envelope under external pressure.

14. The apparatus of claim 1, further comprising a means for supporting the evaporation channel under external pressure.

15. The apparatus of claim 1, further comprising an evaporation manifold disposed in the evaporation channel.

16. The apparatus of claim 1, wherein the evaporation channel comprises at least one side having a textured surface configured to maintain an open flow path under external pressure.

17. The apparatus of claim 1, wherein the evaporation channel comprises at least one side that is embossed.

18. The apparatus of claim 1, wherein the evaporation channel comprises a raised pattern of protrusions configured to support the evaporation channel under external pressure.

19. The apparatus of claim 1, wherein the evaporation channel comprises recessed channels configured to maintain an open flow path under external pressure.

20. The apparatus of claim 1, wherein the evaporation channel further comprises bosses configured to support the evaporation channel under external pressure.

21. The apparatus of claim 1, wherein the evaporation channel is formed at least in part by a cover coupled to the envelope over the vapor-transfer surface.

22. The apparatus of claim 1, further comprising a baffle disposed along a portion of the evaporation channel.

23. The apparatus of claim 1, wherein the evaporation channel is formed at least in part by a cover having edges coupled to the envelope along a length of the vapor-transfer surface.

24. The apparatus of claim 1, wherein the evaporation channel is defined at least in part by a cover having edges and a center portion coupled to the envelope along a length of the vapor-transfer surface.

25. The apparatus of claim 1, further comprising a feedback path substantially parallel to the fluid transfer bridge.

26. The apparatus of claim 25, further comprising a means for supporting the feedback path under external pressure.

27. The apparatus of claim 25, wherein the feedback path comprises at least one side having a textured surface configured to maintain an open flow path under external pressure.

28. The apparatus of claim 25, wherein the feedback path comprises at least one side that is embossed.

29. The apparatus of claim 1, wherein the evaporation channel comprises a return path.

30. The apparatus of claim 1, further comprising a dressing coupled to the first transfer channel.

31. The apparatus of claim 1, further comprising a negative-pressure source coupled to the second transfer channel.

32. The apparatus of claim 1, further comprising:
a negative-pressure source fluidly coupled to the second transfer channel; and
a positive-pressure source fluidly coupled to the evaporation channel.

33. The apparatus of claim 1, further comprising a pump comprising a negative-pressure port and a positive-pressure port, wherein the negative-pressure port is fluidly coupled to the second transfer channel and the positive-pressure port is fluidly coupled to the evaporation channel.

34. The apparatus of claim 1, further comprising:
a negative-pressure source; and
a flow controller configured to selectively couple the negative-pressure source to the fluid transfer bridge or the evaporation channel.

35. The apparatus of claim 1, further comprising:
a pump comprising a negative-pressure port and a positive-pressure port; and
a flow controller configured to selectively couple the negative-pressure port to the second transfer channel or to ambient air;
wherein the positive-pressure port is fluidly coupled to the evaporation channel.

36. The apparatus of claim 1, further comprising a manual pump coupled to the second transfer channel.

37. An apparatus for treating a tissue site with negative pressure, the apparatus comprising:
an envelope defining a fluid chamber and comprising:
a vapor-transfer surface,
a first transfer channel, and
a second transfer channel;
an absorbent in the fluid chamber;
an evaporation channel disposed adjacent to the vapor-transfer surface;
a liquid filter disposed in the second transfer channel;
a pump having a negative-pressure port fluidly coupled to the second transfer channel and a positive-pressure port fluidly coupled to the evaporation channel;
a valve fluidly coupled to the negative-pressure port; and
a controller configured to operate the valve to selectively couple the negative-pressure port and the positive-pressure port to at least one of the fluid chamber, the evaporation channel, and ambient air.

38. The apparatus of claim 37, wherein:
the positive-pressure port is fluidly coupled to the evaporation channel; and
the controller is configured to operate the valve to selectively couple the negative-pressure port to the fluid chamber and to ambient air.

39. The apparatus of claim 37, wherein:
the positive-pressure port is fluidly coupled to ambient air; and
the controller is configured to operate the valve to selectively couple the negative-pressure port to the fluid chamber and to the evaporation channel.

40. The apparatus of any of one of claims 37-39, further comprising:
a means for measuring a pressure difference between a distal end of the fluid chamber and a proximal end of the fluid chamber; and
a means for operating the pump to compensate for the pressure difference.

41. The apparatus of any one of claims 37-39, wherein:
the fluid chamber is an elongated chamber having a distal end and a proximal end; and
the controller is configured to measure a pressure difference between the proximal end and the distal end, and to operate the pump to compensate for the pressure difference.

42. The apparatus of any one of claims 37-41, further comprising a dressing fluidly coupled to the first transfer channel.

43. An apparatus for treating a tissue site with negative pressure, the apparatus comprising:
an envelope comprising a vapor-transfer surface and defining an elongated fluid chamber having a proximal end and a distal end;
a fluid transfer bridge disposed in the fluid chamber; and
a means for measuring pressure adjacent to the distal end of the fluid chamber.

44. The apparatus of claim 43, wherein the means for measuring pressure adjacent to the distal end of the fluid chamber comprises:
a feedback path substantially parallel to the fluid chamber;
a sensor fluidly coupled to the feedback path; and
a controller configured to receive a signal from the sensor indicative of pressure at the distal end of the fluid chamber.

45. The apparatus of claim 43, wherein the means for measuring pressure adjacent to the distal end of the fluid chamber comprises:
a fluid conductor substantially parallel to the fluid chamber;
a sensor fluidly coupled to the fluid conductor; and
a controller configured to receive a signal from the sensor indicative of pressure at the distal end of the fluid chamber.

46. The apparatus of claim 45, further comprising a means for supporting the fluid conductor under external pressure.

47. The apparatus of claim 45, wherein the fluid conductor comprises a textured surface configured to support the fluid conductor under external pressure.

48. The apparatus of claim 45, wherein the fluid conductor comprises an embossed surface configured to support the fluid conductor under external pressure.

49. The apparatus of any one of claims 44-48, wherein the controller is further configured provide an indicator if the pressure exceeds a threshold pressure.

50. The apparatus of claim 43, further comprising an evaporation channel disposed adjacent to the vapor-transfer surface.

51. The apparatus of claim 50, further comprising:
a negative-pressure source fluidly coupled to the fluid chamber; and
a positive-pressure source fluidly coupled to the evaporation channel.

52. The apparatus of claim 51, wherein the controller is further configured to:
receive a signal indicative of pressure at the proximal end of the fluid chamber;
determine a difference between pressure at the proximal end and the distal end; and
activate the positive-pressure source if the difference is exceeds a threshold difference.

53. The apparatus of claim 51, wherein the controller is further configured to:
receive a signal indicative of a saturation level in the fluid transfer bridge; and
activate the positive-pressure source to maintain the saturation level within a saturation band.

54. The use of the apparatus of any preceding claim to treat a tissue site with negative pressure.

55. The use of the apparatus of any preceding claim to treat a tissue site with negative pressure, wherein a decrease in negative pressure is less than 0.05 mmHg/millimeter to the tissue site over a treatment period.

56. The use of claim 55, wherein the treatment period is at least one day.

57. The use of claim 55, wherein the treatment period is between one day and seven days.

58. The use of the apparatus of any of claims 1-36 to treat a tissue site with negative pressure, wherein a pressure change between a first end of the fluid transfer bridge and a second end of the fluid transfer bridge is less than 25 mmHg over a treatment period.

59. The use of claim 58, wherein the treatment period is at least one day.

60. The use of claim 58, wherein the treatment period is at least one day and up to at least seven days.

61. The use of the apparatus of any of claims 37-42 to treat a tissue site with negative pressure, wherein a pressure change between the pump and the tissue site through the second transfer channel is less than 25 mmHg over a treatment period.

62. The use of claim 61, wherein the treatment period is at least one day.

63. The use of claim 61, wherein the treatment period is at least one day and up to at least seven days.

64. The systems, apparatuses, and methods substantially as described herein.
